(19) United States
(12) Patent Application Publication
(10) Pub. No.: US 2011/0286608 A1
(43) Pub. Date: Nov. 24, 2011

(54) EARPIECE COMMUNICATION SYSTEM

(75) Inventors: Olivier Hautier, Savagnier (CH); Rainer Platz, Colombier (CH)

(73) Assignee: PHONAK AG, Staefa (CH)

(21) Appl. No.: 13/145,182

(22) PCT Filed: Jan. 21, 2009
(86) PCT No.: PCT/EP2009/050672

§ 371 Date: Jul. 19, 2011

(51) Int. Cl. A61F 11/06; H04R 1/10

(52) U.S. Cl. 381/72; 381/74

(57) ABSTRACT

A communication system is provided having at least one earpiece (14, 114, 214) including a loudspeaker (26) for stimulating a user's hearing according to audio signals supplied to the earpiece; a voice microphone arrangement (28, 58, 60, 62, 258, 260) for picking up the user's voice; a configurable audio interface (22, 32, 34, 36, 38) for exchanging audio signals with an external wireless communication device (24) selected from a plurality of different types of external wireless communication devices; and a configuration interface (40) for adapting the configuration of the audio interface to the selected type of the external wireless communication device by an external configuration device (18).

### BACKGROUND OF THE INVENTION

1. Field of the Invention

The invention relates to a communication system comprising at least one earpiece including a loudspeaker, a voice microphone arrangement for picking up the voice of the earpiece user, and an audio interface for exchanging audio signals with an external wireless communication device, such as a radio or a mobile phone.

2. Description of Related Art

Different types of such communication systems are known. Usually, the audio interface is included in a unit which is to be worn at the user's body, for example at a neck loop.

International Patent Application Publication WO 2008/052575 A1 relates to a communication system which comprises a transmission unit which is worn at a neck loop and which includes the voice microphone arrangement; a transmission unit is provided for transmitting audio signals via an inductive link to the earpiece, which inductive link is an amplitude modulation inductive link having a carrier frequency of from 100 kHz to 30 MHz.

International Patent Application Publication WO 2007/073818 A1 and U.S. Patent Application Publication 2007/0160243 A1 relate to communication systems, wherein the voice microphone arrangement is provided as part of the earpiece and comprises a first microphone oriented acoustically outwardly towards the environment and a second microphone oriented acoustically inwardly towards the user's ear canal and wherein the audio interface comprises an audio signal processing unit for processing audio signals from the first and second microphone by a blind source separation (BSS) algorithms adapted to separate the user's voice from ambient sound.

International Patent Application Publication WO 2007/082579 A2 and corresponding U.S. Patent Application Publication 2010/0119077 A1 relate to a communication system, wherein the ear pieces are designed as hearing protection earplugs and wherein each earplug comprises a microphone for picking up ambient sound, which, depending on the ambient noise level, may be supplied via an audio signal processing unit worn at the user's body to the loudspeaker of the respective ear piece. A boom microphone is provided at one of the ear pieces to pick up the user's voice; alternatively, each of the ear pieces may be provided with a second, internal microphone, in order to separate the user's voice from ambient sound by a BSS algorithm carried-out in the audio signal processing unit. Typically, such communication systems are powered by the external wireless communication device, i.e., the radio or the mobile phone and the audio interface is connected via a plug connector to the external wireless communication device. One problem encountered by such communication devices is that there is a huge variety of radio terminals available on the market, for which the communication system needs to be adjusted. In particular, there is no standardization of the interface for the connection to the radio terminal, so that fundamental parameters such as the power supply voltage, the noise level on the power supply voltage, the audio input sensitivity and the audio output level vary largely between different types of radio terminals. For example, power supply levels provided by such radio terminals may vary between 2 V and 28 V. Also, mechanisms for automatic peripheral device detection by, e.g., sensing of resistors varies from one radio device to another.

Under these circumstances, the present practice is to specifically configure the communication system to the specific radio terminal used by the customer by specific hardware modifications. Usually there is no possibility to modify the configuration once the communication system has been finalized, i.e., has been adapted to the specific radio terminal. As a consequence, the customer's invest with regard to the communication system is basically lost, if he decides to change his radio terminals.

U.S. Patent Application Publication 2007/0098195 A1 relates to a hearing aid headset which is programmable via a wireless interface by a computer, whereby a specific hearing aid profile may be selected from a plurality of profiles.

### SUMMARY OF THE INVENTION

It is an object of the invention to provide for a communication system which can be easily used for different types of external wireless communication devices, such as radio terminals.

According to the invention, this object is achieved by a communication system and by a communication arrangement as described herein.

The invention is beneficial in that the configuration of the audio interface of the communication system can be easily adapted to the specific type of external wireless communication device which is to be used together with the communication system. In accordance with one embodiment, a configuration interface is provided for adapting the configuration of the audio interface to the specific type of external wireless communication device by an external configuration device. Thereby, the configuration of the audio interface can be adapted by the manufacturer, the distributor or the actual user.

In accordance with another embodiment, the communication system is provided with means for recognizing the specific type of external wireless communication device to which the audio interface is connected and with means for adapting the configuration of the audio interface accordingly. Thereby, an automatic adaptation to the specific type of external wireless communication device can be achieved.

In accordance with another feature of the invention, the ability to configure the audio interface is used to adapt an identifier so as to present the “correct” identifier expected by the external wireless communication device when the communication system is connected to the external wireless communication device. Usually, external wireless communication devices have means to detect the connection of an accessory, such as a communication system. This is done typically by sensing the presence of pre-defined resistor values or short circuits on one or several input lines to the device. In the present case, the identifier may be, for example, a varistor or a resistor bench, which is set by the communication system to the resistance value expected by the external wireless communication device.

Preferably, at least one of the following parameters of the audio interface is configurable via the configuration interface: the gain applied to the audio signals received from the external wireless communication device, the gain applied to the voice microphone arrangement signal supplied to the external wireless communication device, and the supply voltage transformation ratio (as already mentioned, the communication system is usually powered by the external wireless communication device).

Usually, the audio interface comprises an audio signal processing unit for processing the audio signals received from the external wireless communication device and the audio signals supplied to the external wireless communication device, which audio signal processing unit preferably is configurable by the configuration interface for individually adapting the configuration of the audio signal processing unit to the user preferences. For example, the following parameters of the audio signal processing unit may be configurable via the configuration interface: output level of the voice microphone arrangement signal; output level, attack time constant and/or release time constant of an automatic gain control (AGC) mechanism; threshold level, attack time constant and/or release time constant of a voice controlled operation (VOX) mechanism; level and shape of acoustic acknowledge signals to the user; enabling/disabling of voice microphone arrangement signal; enabling/disabling of mute functionality; and enabling/disabling of remote control functionality.

The configuration interface may be designed as a wireless interface to a remote control device for controlling the communication. The configuration interface may be wireless or it may comprise a plug connection to the configuration device. Usually, the configuration device will be a computer running configuration software.

Usually, each earpiece will be designed to be worn at least in part in the user's ear canal. However, the earpiece, in principle, also may be designed to be worn at the user's ear as part of a headphone.

These and further objects, features and advantages of the present invention will become apparent from the following description when taken in connection with the accompanying drawings which show several embodiments in accordance with the present invention for purposes of illustration only.

### BRIEF DESCRIPTION OF THE DRAWINGS

FIG. 1 is a block diagram of a first embodiment of a communication system according to the invention,

FIG. 2 is a block diagram of an alternative embodiment of a communication system according to the invention;

FIG. 3 is a block diagram of a further embodiment of a communication system according to the invention; and

FIG. 4 is a view of an embodiment of a communication system according to the invention.

### DETAILED DESCRIPTION OF THE INVENTION

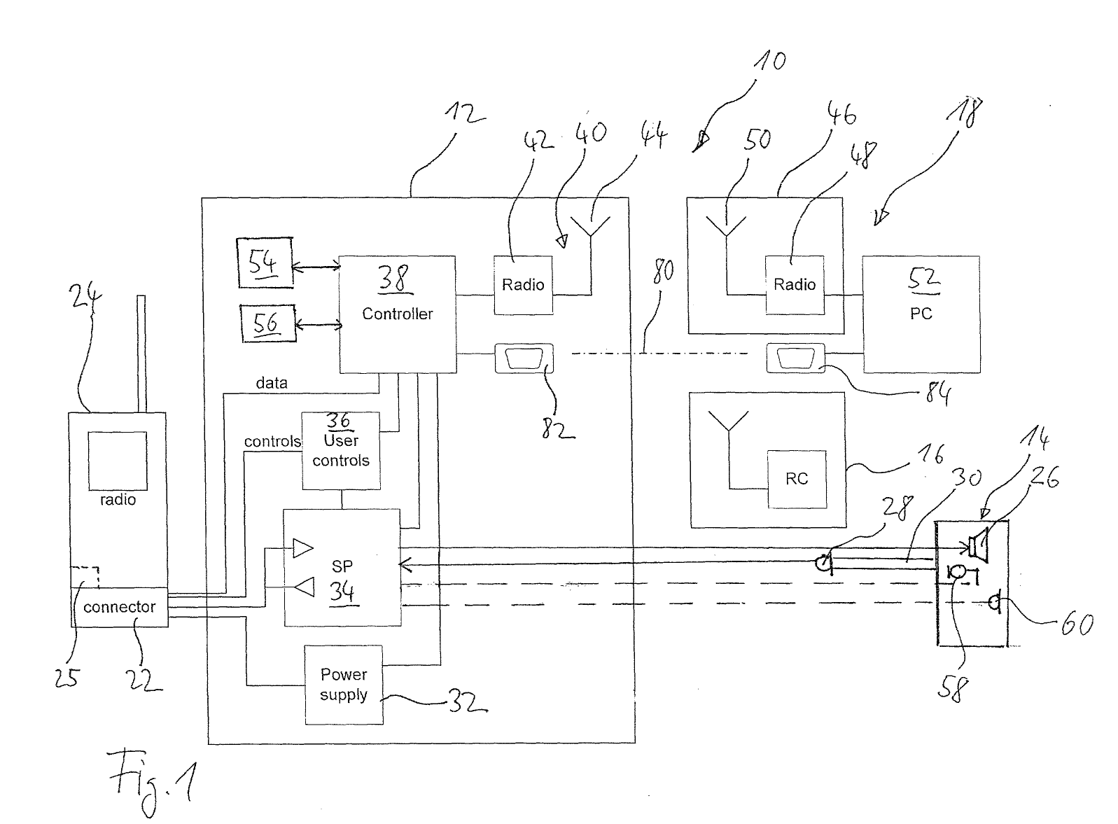

FIG. 1 is a block diagram of an example of a communication system, wherein in addition some modifications are shown in dashed lines.

The communication system 10 comprises a central unit 12, which usually will be designed to be worn at a user's body below the neck, for example, with a neck loop (not shown), at least one earpiece 14, which usually is to be worn at least in part in the user's ear canal (in most cases, the system will include two of such earpieces 14, one for each ear), a remote control 16, an external configuration device 18, and a connector 22 for connecting the central unit 12 to an external wireless communication device 24 which usually will be a radio device.

The earpiece 14 comprises a loudspeaker 26 for supplying sound into the user's ear canal. According to one embodiment, one of the earpieces 14 may be provided with a boom microphone 28 which is attached to the earpiece 14, usually by a flexible bow 30, for capturing the user's voice.

The central unit 12 comprises a power supply 32, an audio signal processing unit 34, a user control unit 36, a controller 38 and a wireless configuration interface 40 comprising a radio transmitter 42 and an antenna 44.

The power supply 32 usually is powered by the radio device 24 and comprises means for transforming the supply voltage of the radio device 24 to the voltage level needed in the central unit 12.

The audio signal processing unit 34 is provided for supplying the audio signals received from the radio device 24 via the connector 22 to the speaker 26 in order to stimulate the user's hearing with the voice of another person using a radio device communicating with the radio device 24 or a communication system connected to such radio device. In addition, the audio signal processing unit 34 is provided for supplying audio signals received from the microphone 28 to the radio device 24 via the connector 22 in order to provide the user's voice to another person via the radio device 24. The audio signal processing unit 34 is equipped with the necessary filters and amplifiers and may particularly also comprise a digital signal processing (DSP) unit.

The user control unit 36 serves as a manual user interface in order to control operation of the audio signal processing unit 34 and the radio device 24 and may include the following functions: on/off, audio signal processing mode (program) change, PTT (push-to-talk), and call accept/reject or voice dialing (if the communication system 10 is connected to a mobile phone). The same commands or a subset thereof may be provided on the remote control 16.

The controller 38 is provided for controlling operation of the user control unit 36, the audio signal processing unit 34 and the power supply 32. In addition, the controller 38 may exchange data with the radio device 24 via the connector 22. The controller 38 receives data/commands from the configuration interface 40 which communicates via a wireless link with a transmission unit 46 of the configuration device 18 comprising a transmitter or a transceiver 48 and an antenna 50. The configuration device 18 also includes a computer 52 running configuration software for adapting the configuration of the central unit 12 to the specific type of radio device 24. The audio signal processing unit 34, the power supply 32, the user control unit 36 and the controller 38 form a configurable audio interface for exchanging audio signals with the radio device 24 which serves as an external wireless communication device. Thus, the configuration interface 40, by acting on the controller 38, serves to adapt the configuration of the audio interface to the specific type of external wireless communication device.

The configuration interface 40 may be used to adapt the gain applied to the audio signals received from the radio device 24 and the gain applied to the audio signals from the microphone 28, which are supplied to the radio device 24, according to the specific type of radio device 24. Moreover, the configuration interface 40 may be used to adapt the supply voltage transformation ratio in the power supply 32 to the voltage level supplied by the specific radio device 24. The audio signal processing unit 34 may comprise an AGC-mechanism and/or a VOX-mechanism, and it may be used to supply acoustic acknowledge signals to the user via the speaker 26.

The configuration interface 40 and the controller 38 may be used to adapt the audio signal processing unit 34 individually to the user preferences. For example, the following parameters of the audio signal processing unit 34 may be configured via the configuration interface 40: the output level of the microphone signal, the output level, the attack time constant and/or the release time constant of the AGC mechanism, the threshold level, attack time constant and/or release time constant of the VOX mechanism, the level and shape of acoustic acknowledge signals, enabling/disabling of the microphone signal, enabling/disabling of mute functionality, and enabling/disabling of remote control functionality.

In the system shown in FIG. 1, the wireless configuration interface 40 serves to communicate not only with the configuration device 18, but also with the remote control 16, i.e., the configuration interface 40 receives remote control demands 16 and supplies it to the controller 38.

In addition to the elements mentioned above, the central unit 12 also may comprise a data memory 54 and a program memory 56. The data memory 54 may be used for storing user data, service data, use history data and access codes, and the data memory 54 may be writeable via the configuration interface 40, so that these parameters can be configured by the configuration device 18. The program memory 56 serves to store the program by which the controller 38 is operated, and it may be writeable via the configuration interface 40 for carrying-out a software or firmware update initiated by the configuration device 18.

According to a modified embodiment, the earpiece 14 may be designed as a hearing protection ear plug. In addition, the earpiece 14 may be provided with a microphone 58 for capturing ambient sound at the ear level, wherein the audio signals captured by the microphone 58 are supplied to the audio signal processing unit 34, which applies a level dependent gain to these audio signals, with the amplified audio signals being supplied to the speaker 26 of the earpiece 14 in order to provide for an active hearing protection function of the earpiece 14.

Usually, the speaker 26, the boom microphone 28 and the earpiece microphone 58 are connected by wire to the audio signal processing unit 34.

According to another modified embodiment, the boom microphone 28 may be replaced by an arrangement wherein the earpiece 14 (or both earpieces 14) comprises a first microphone 58 oriented acoustically outwardly towards the environment and a second microphone 60 oriented acoustically inwardly towards the user's ear canal, wherein the audio signal processing unit 34 is designed for processing audio signals supplied from the first microphone 58 and second microphone 60 by a BSS-algorithm adapted to separate the user's voice from ambient sound. Such an arrangement is described, for example, in International Patent Application Publication WO 2007/073818 A1. Also, in this case, the microphones 58, 60 and the speaker 26 usually are connected by a wire to the audio signal processing unit 34.

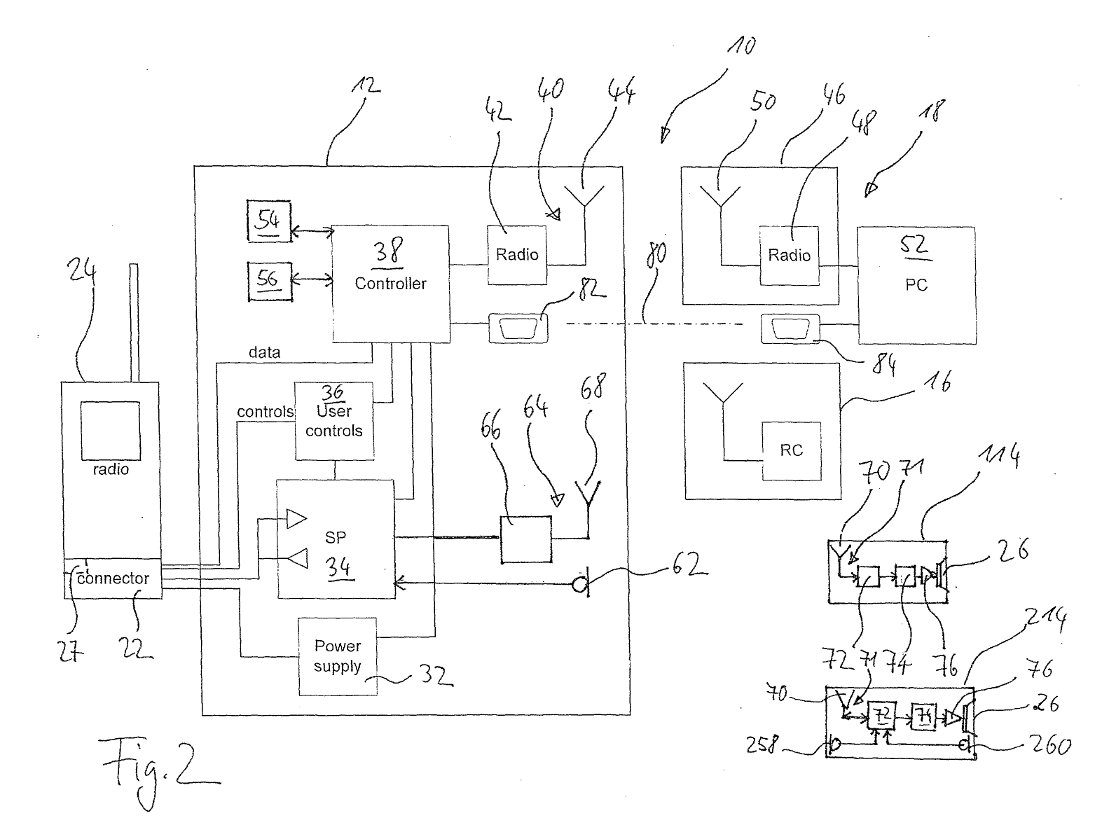

An alternative embodiment of a communication system according to the invention is shown in FIG. 2, wherein the wired connection between the audio signal processing unit 34 and the earpieces 114 is replaced by a wireless link. A further modification is that the earpiece 114 of FIG. 2 neither comprises a microphone nor is connected with a boom microphone. Rather, according to FIG. 2, the central unit 12 comprises a microphone 62 for capturing the user's voice.

The audio signal output of the audio signal processing unit 34, i.e., the audio signals received from the radio device 24 and processed by the audio signal processing unit 34, are supplied to a transmission unit 64 comprising a transmitter 66 and an antenna 68 for transmitting the processed audio signals to a receiver unit 71 of the earpiece 114 comprising an antenna 70 and a receiver 72. Preferably, the link between the transmission unit 64 and the receiver unit 71 is an amplitude modulation (AM) inductive link having a carrier frequency of from 100 kHz to 30 MHz, in which case the transmitter 66 is an AM modulator, and the receiver 72 is a corresponding AM demodulator. The received demodulated audio signals are supplied to an audio processing unit 74 of the ear piece 114 and from there to a power amplifier 76 which drives the speaker 26.

According to a modified embodiment, the element 66 may be designed as a transceiver rather than as a transmitter, so that the audio signal processing unit 34 of the central unit 12 may not only send audio signals to earpiece 114 but also may receive audio signals from a microphone arrangement, such as microphones 258, 260, of the earpiece 214. In this case, the element 72 of the earpiece will be designed as a transceiver rather than as a receiver. The wireless earpiece 214 then may serve the same purposes as the wired earpiece 14, namely to also pick up the user's voice by the microphones 158, 160 and to supply it to the audio signal processing unit 34 wherein, for example, a BSS algorithm may applied to separate the user's voice from ambient sound.

Alternatively, the BSS algorithm may be applied in the earpiece 214 if the element 72 comprises signal processing capabilities.

The bidirectional wireless link between the central unit 12 and the earpiece may be implemented by using analog radio technologies between 100 and 1000 MHz, digital magnetic/inductive bidirectional radio technologies between 2 and 20 MHz, digital bidirectional narrow band radio technologies between 200 MHz and 6 GHz or digital bidirectional ultra wide band radio technologies between 3 and 20 GHz.

According to a further modified embodiment, the central unit 12 both according to FIGS. 1 & 2 may include means for recognizing the type of external wireless communication device, i.e., the type of radio device 24, to which the connector 22 of the communication system is connected to. To this end, the radio device 24 may be provided with an identifier 25 (as indicated in FIG. 1), such as at least one specific resistor, at the interface to the connector 22 so that the type of radio device can be identified by the central unit 12 by measuring the resistance at one pin or several pins of the connector 22. Alternatively, since the type of connector 22 usually is specific to the type of radio device 24, the connector 22 itself may be provided with an identifier 27 (as indicated in FIG. 2) so that the central unit may identify the type of connector 22 which is connected to the central unit 12, thereby identifying the type of radio device 24. The controller 38 is designed to adapt the configuration of the audio interface, i.e., the user control unit 36, the audio signal processing unit 34 and the power supply 32, to the recognized type of external wireless communication device. To this end, the controller 38 may communicate with the external configuration device 18 via the wireless link. However, for such auto-identification embodiments the external configuration device 18 is optional, since all information necessary to adapt the configuration of the audio interface to the recognized type of external communication device may be implemented in the central unit.

According to a further modified embodiment, the wireless link between the central unit 12 and the configuration unit 18 may be replaced by a corresponding wired connection 80, wherein both the central unit 12 and the external configuration device 18 are provided with corresponding connectors 82 and 84, respectively.

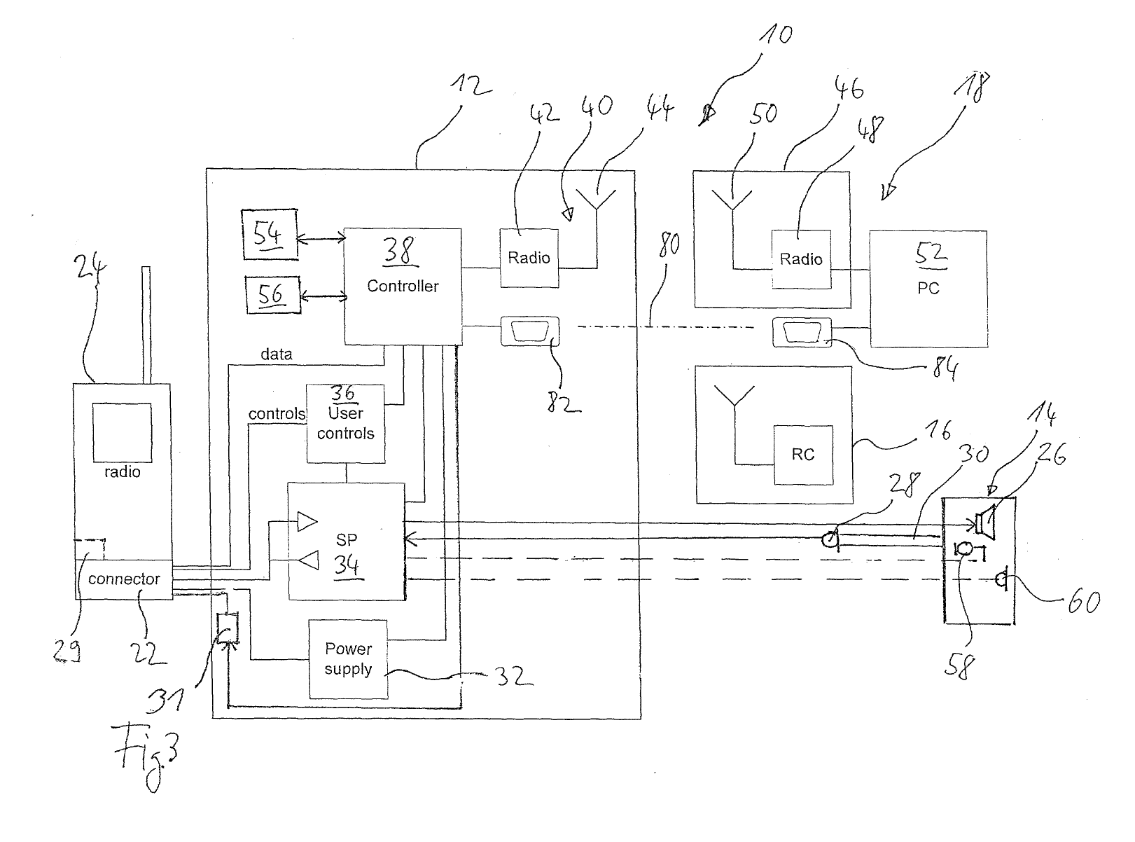

In FIG. 3, an embodiment is shown, wherein the ability to configure the audio interface is used to adapt an identifier of the communication system 10 so as to present the “correct” identifier expected by the external wireless communication device 24 when the communication system is connected to the external wireless communication device 24. This is beneficial because external wireless communication devices 24 usually have a means (indicated at 29 in FIG. 3) to detect the connection of an accessory device, such as a communication system 10. This is done typically by sensing the presence of pre-defined resistor values or short-circuits on one or several input lines to the accessory device.

In the example shown in FIG. 3, the central unit 12 is provided with an identifier 31 which is controlled by the controller 38. The identifier 31 may be, for example, a varistor or a resistor bench, which is set by the controller 38 to the resistance value expected by the external wireless communication device 24. To this end, the controller 38 needs to know which type of external wireless communication device 24 is connected to the communication system 10. Such information may be provided by the configuration device 18, or, if available, by the identifier 25 (FIG. 1) or 27 (FIG. 2). When the identifier 31 has been set to the correct value, the external wireless communication device 24 is able to recognize the communication system 10 as being connected as an accessory device.

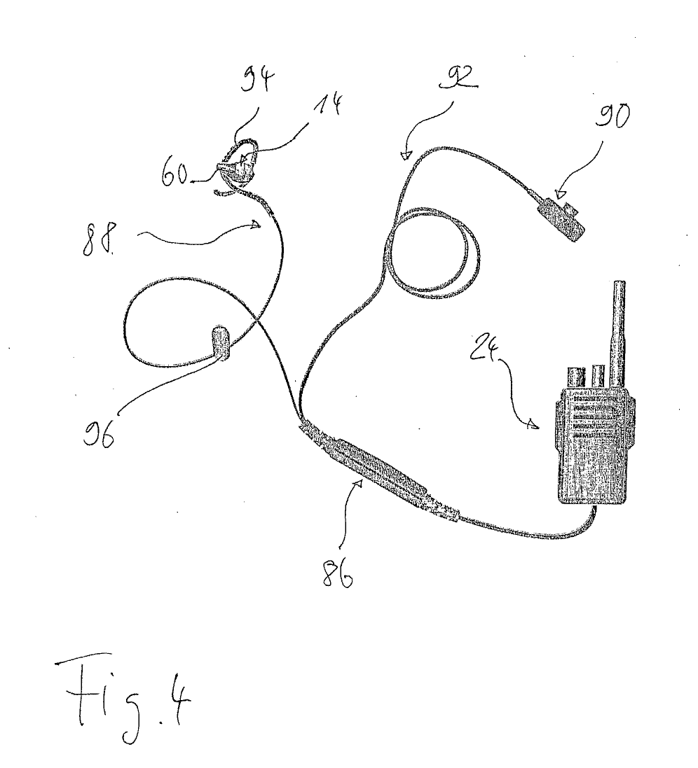

In FIG. 4, a practical example of a BSS communication system according to FIG. 1 is shown, wherein the earpiece 14 is an earplug and the acoustically inwardly oriented microphone 60 is a bone conduction microphone, i.e., it is adapted for picking up the user's voice via bone conduction from the user's skull (to this end, the microphone 60 abuts the wall of the ear canal when the earpiece is worn by the user). The earpiece 14 is mechanically and electrically connected to a cable assembly 88 which leads to a housing 86 containing the central unit 12. The cable assembly 88 is surrounded at an end portion facing the earpiece 14 by a fastening sleeve 94 having a bent shape and being made of a plastics material. The fastening sleeve 94 can be moved along the cable assembly 88 in order to enable the user to find an optimum position of the fastening sleeve 94. The fastening sleeve 94 is designed to form a suspension loop around the upper part of the user's pinna. The cable assembly 88 also comprises a clip 96 for fixing the cable assembly 88 somewhere at the user's clothing.

The system also includes a push-to-talk (PTT) button 90 which is connected via a cable 92 to the housing 86 and from there to the user control unit 36 in order to control the transmitter/receiver of the communication device 24.

While various embodiments in accordance with the present invention have been shown and described, it is understood that the invention is not limited thereto, and is susceptible to numerous changes and modifications as known to those skilled in the art. Therefore, this invention is not limited to the details shown and described herein, and includes all such changes and modifications as encompassed by the scope of the appended claims.

1. A communication system, comprising:
at least one earpiece including a loudspeaker for stimulating a user's hearing according to audio signals supplied to the earpiece;
a voice microphone arrangement for picking up the user's voice;
a configurable audio interface for exchanging audio signals with an external wireless communication device selected from a plurality of different types of external wireless communication devices; and
a configuration interface for adapting the configuration of the audio interface to a selected type of the external wireless communication device by an external configuration device.

2. The system of claim 1, wherein the communication system is adapted to be powered by the external wireless communication device.

3. The system of claim 2, wherein at least one of the following parameters of the audio interface is configurable via the configuration interface: a gain applied to audio signals received from the external wireless communication a gain applied to audio signals of the voice microphone arrangement supplied to the external wireless communication device, and a transformation ratio applied to a supply voltage from the external wireless communication device.

4. The system of claim 1, wherein the audio interface comprises an audio signal processing unit for processing the audio signals received from the external wireless communication device and the audio signals supplied to the external wireless communication device.

5. The system of claim 4, wherein the audio signal processing unit is configurable by the configuration interface for individually adapting a configuration of the audio signal processing unit to user preferences.

6. The system of claim 5, wherein at least one of the following parameters of the audio signal processing unit is configurable via the configuration interface: an output level of a voice microphone arrangement signal; at least one of an output level, an attack time constant and a release time constant of an automatic gain control mechanism; at least one of a threshold level, an attack time constant and an release time constant of a voice-controlled operation mechanism; a level and a shape of acoustic acknowledge signals supplied to loudspeakers; enabling/disabling of voice microphone arrangement signal; enabling/disabling of mute functionality; and enabling/disabling of a remote control functionality.

7. The system of claim 1, further comprising a data memory for storing at least one of user data, service data, use history data, and access codes, and which data memory is writeable via the configuration interface.

8. The system of claim 1, wherein further comprising a program memory for a microcontroller of the communication system, which program memory is writeable via the configuration interface for carrying out a software/firmware update.

9. The system of claim 1, wherein the configuration interface is wireless.

10. The system of claim 9, wherein the configuration interface is a wireless interface to a remote control device for controlling the communication system.

11. The system of claim 9, wherein the external configuration device comprises a computer running configuration software.

12. The system of claim 11, wherein the computer comprises an USB-dongle for wireless communication with the configuration interface.

13. The system of claim 1, wherein the audio interface comprises a plug connector adapted for connection to the external wireless communication device.

14. The system of claim 1, wherein the external wireless communication device is a radio device or a mobile phone.

15. The system of claim 1, wherein the audio interface and the configuration interface form part of a body-worn central unit.

16. The system of claim 4, wherein the earpiece comprises a hearing protection earplug with an earpiece microphone supplying audio signals to the audio signal processing unit which applies a level dependent gain to the supplied audio signals, with the amplified audio signals being supplied to a speaker of the earpiece in order to provide for a active hearing protection function.

17. The system of claim 16, wherein the voice microphone arrangement is a boom microphone attached to the earpiece.

18. The system of claim 1, further comprising a transmission unit to be worn at a user's body for transmitting audio signals, via an inductive link, received from the external wireless communication device, wherein the earpiece includes a receiver unit for receiving audio signals via said inductive link from the transmission unit and a unit for processing audio signals received by the receiver unit, wherein said inductive link is an amplitude modulation inductive link having a carrier frequency of from 100 kHz to 30 MHz.

19. The system of claim 1, further comprising a transceiving unit to be worn at a user's body for transmitting and receiving audio signals, via a radio link, received from or transmitted to the external wireless communication device, wherein the earpiece includes a transceiver unit for receiving and transmitting audio signals via said radio link from the transceiving unit and a unit for processing audio signals received by or to be transmitted from the transceiving unit wherein the earpiece comprises the voice microphone arrangement.

20. The system of claim 18, wherein the audio interface, the configuration interface, the voice microphone arrangement and the transmission unit form part of a body-worn central unit.

21. The system of claim 4, wherein the earpiece comprises a first microphone oriented acoustically outwardly towards an area external to the user's ear and a second microphone oriented acoustically inwardly towards a user's ear canal, wherein the audio signal processing unit is adapted for processing audio signals from the first and second microphone by a blind source separation algorithm adapted to separate the user's voice from ambient sound, the first and second microphone forming the voice microphone arrangement.

22. A communication system, comprising:
at least one earpiece including a loudspeaker for stimulating a user's hearing according to audio signals supplied to the earpiece;
a voice microphone arrangement for picking up a user's voice;
a configurable audio interface for exchanging audio signals with an external wireless communication device selected from a plurality of different types of external wireless communication devices;
means for recognizing the type of external wireless communication device selected from among said plurality of different types of external wireless communication devices to which the audio interface is connected; and
means for adapting a configuration of the audio interface to a recognized type of the external wireless communication device.

23. The system of claim 22, wherein the external wireless communication device is provided with an electronic identifier specific to the type of external wireless communication device.

24. The system of claim 22, wherein an interface which is specific to the type of external wireless communication device is provided with an electronic identifier specific to the type of interface selected from among said plurality of different types of external wireless communication devices.

25. The system of claim 23, wherein the electronic identifier is at least one resistor.

26. A communication arrangement, comprising:
an external wireless communication device selected from a plurality of different types of external wireless communication devices;
and a communication system comprising at least one earpiece including a loudspeaker for stimulating a user's hearing according to audio signals supplied to the earpiece, a voice microphone arrangement for picking up a user's voice, and a configurable audio interface for exchanging audio signals with the external wireless communication device;
wherein external wireless communication device comprises means for recognizing a type of device to which the external wireless communication device is connected; and wherein the communication system comprises means for adapting a configuration of the audio interface to the selected type of external wireless communication device in such a manner that the recognizing means are enabled to recognize the communication system as being connected via the audio interface to the external wireless communication device.

27. The arrangement of claim 26, wherein the means for adapting the configuration of the audio interface to the type of the external wireless communication device comprise a configuration interface controlled by an external configuration device.

28. The arrangement of claim 26, wherein the audio interface comprises an identifier whose electrical resistance is selectable according to the selected type of external wireless communication device and which identifier is sensed by the external wireless communication device when it is connected to the communication system.

29. The arrangement of claim 28, wherein the identifier is resistor bench or a varistor.

30. Use of the communication system of claim 1, comprising: selecting a type of external wireless communication device, and setting, by using the external configuration device, via the configuration interface, the configuration of the audio interface according to the selected type of external wireless communication device.
